(19) United States
(12) Patent Application Publication
(10) Pub. No.: US 2019/0388020 A1
(43) Pub. Date: Dec. 26, 2019

(54) System and Method for Treating and Preventing Cognitive Disorders

(71) Applicant: NeuroPlus Inc., Durham, NC (US)

(72) Inventors: Jacob Stauch, Durham, NC (US); Jeroen Kools, Campbell, CA (US)

(21) Appl. No.: 16/447,013

(22) Filed: Jun. 20, 2019

Related U.S. Application Data
(60) Provisional application No. 62/687,553, filed on Jun. 20, 2018.

(51) Int. Cl. A61B 5/00; A61B 5/16; A61N 1/04; A61B 5/04

(52) U.S. Cl. CPC A61B 5/4088; A61B 5/165; A61N 1/0404; A61B 5/04004; A61B 5/486; A61B 5/6803; A61B 5/7207; A61B 5/4812; A61B 5/6814

(57) ABSTRACT

A computer system is configured to present an individual having Alzheimer's disease or other form of dementia by stimulating the individual's brain using one or both of sensory stimulation and an electrical current. The individual's brain may be stimulated while the individual is awake, or asleep. In situations where the individual is asleep, the computer system may also determine the individual's sleep-stage. Stimulating the individual's brain induces gamma frequency activity in the individual's brain. The gamma frequency activity is captured and used to provide real-time feedback in a loop to the user regarding their brain's oscillatory activity to enhance the entrainment of the gamma frequency by the sensory stimulation and/or the electrical current.

### RELATED APPLICATIONS

This application claims priority to U.S. Application Ser. No. 62/687,553, filed Jun. 20, 2018, the disclosure of which is incorporated in its entirety by reference herein.

### FIELD OF DISCLOSURE

The present disclosure relates to computer training systems, and more particularly, to computer systems configured to treat individuals, such as patients, with a cognitive dysfunction.

### BACKGROUND

Alzheimer's disease (AD), also referred to as “Alzheimer's,” is a chronic neurodegenerative disease suffered by over 25 million people worldwide. Generally, most people afflicted with AD do not experience symptoms of the disease until they are over 65 years old. A minority of those afflicted with AD will experience the symptoms before age 65. These younger people are said to suffer from early-onset AD.

The cause of AD is not well understood, but there are some suspected risk factors. Mostly, the risk is believed to be genetic, although other factors can include head injuries and depression. However, individuals exhibiting the symptoms of AD generally have difficulty remembering important events that have occurred, as well as the people in their lives (including family and lifelong friends). Additionally, people afflicted with AD have problems with language, become easily disoriented, and in many cases, develop behavioral problems.

### SUMMARY

Embodiments of the present disclosure provide a system and method that enhances the effect of gamma frequency entrainment by stimulating an individual's brain using sensory stimulation (i.e., a steady-state stimulus at one or more modalities), an electrical current, or both. As defined herein, “gamma frequency” is a frequency band at which some neural oscillatory activity in a human brain occurs. The gamma frequency band is usually considered to extend from about 30 Hz to about 100 Hz. The system and method described herein is beneficial for treating individuals with various forms of dementia such as Alzheimer's disease, for example, and may be used to treat individuals while they are awake or during sleep.

In one embodiment, the present disclosure provides a method for treating an individual with a cognitive disorder. In this embodiment, the method comprises stimulating an individual's brain using one or both of sensory stimulation and an electrical current. Further, while the individual's brain is being stimulated, the method comprises receiving signals indicating gamma frequency oscillatory activity in the individual's brain induced by the stimulation, generating feedback information based on the signals indicating the gamma frequency oscillatory activity in the individual's brain, outputting the feedback information in a feedback loop, and enhancing entrainment of gamma frequency induced by the stimulation in the individual's brain responsive to the feedback information in the feedback loop.

The present disclosure also provides a computing device for treating an individual with a cognitive disorder. In one embodiment, the computing device comprises communications interface circuitry and processing circuitry. The communications interface circuitry is configured to communicate with an Electroencephalography (EEG) device attached to an individual being treated for a cognitive disorder. The processing circuitry is operatively coupled to the communications interface circuitry, and configured to stimulate an individual's brain using one or both of sensory stimulation and an electrical current. While the individual's brain is being stimulated, the processing circuit is configured to receive signals indicating gamma frequency oscillatory activity in the individual's brain induced by the stimulation, generate feedback information based on the signals indicating the gamma frequency oscillatory activity in the individual's brain, output the feedback information in a feedback loop, and enhance entrainment of gamma frequency induced by the stimulation in the individual's brain responsive to the feedback information in the feedback loop.

The present disclosure also provides a non-transitory computer-readable medium having a control application program stored thereon that, when executed by processing circuitry of a computing device, causes the computing device to stimulate an individual's brain using one or both of sensory stimulation and an electrical current. Additionally, while the individual's brain is being stimulated, the control application also configures the computing device to receive signals indicating gamma frequency oscillatory activity in the individual's brain induced by the stimulation, generate feedback information based on the signals indicating the gamma frequency oscillatory activity in the individual's brain, output the feedback information in a feedback loop, and enhance entrainment of gamma frequency induced by the stimulation in the individual's brain responsive to the feedback information in the feedback loop.

### BRIEF DESCRIPTION OF THE DRAWINGS

FIG. 1 is a perspective view illustrating a system configured according to one embodiment of the present disclosure.

FIG. 2 is a flow diagram illustrating a method for treating individuals with Alzheimer's disease according to one embodiment of the present disclosure.

FIG. 3 is a functional block diagram illustrating some functional components of an EEG device and a computing device configured to treat individuals with Alzheimer's disease according to one embodiment of the present disclosure.

FIG. 4 is a perspective view illustrating a system configured for treating a sleeping individual in accordance with embodiments of the present disclosure.

FIG. 5 is a perspective view illustrating a smart sleep mask configured for treating a sleeping individual according to one embodiment of the present disclosure.

FIG. 6 is a flow diagram illustrating a method for treating an individual with Alzheimer's disease while they are sleeping according to one embodiment of the present disclosure.

FIG. 7 is a flow diagram illustrating another method for treating an individual with Alzheimer's disease according to one embodiment of the present disclosure.

FIG. 8 is a functional block diagram illustrating functional components of an EEG device, a smart sleep mask, and a computing device configured to treat a sleeping individual according to one embodiment of the present disclosure.

### DETAILED DESCRIPTION

The present disclosure provides a system and method for treating and preventing cognitive diseases, such as Alzheimer's disease, through the entrainment of the human brain to produce gamma activity. More specifically, embodiments of the present disclosure output one or both of sensory stimulation and an electrical current to an individual (e.g., a patient being treated for Alzheimer's disease) in order to, respectively, indirectly or directly stimulate the individual's brain into entering a specific gamma activity state. During operation, the present embodiments may present only a single sensory stimulus or electrical current to the individual, or multiple different sensory stimuli and/or the electrical current to the individual. Regardless, stimulating the individual's brain according to the present disclosure elicits a “frequency following” response from the brain, thereby encouraging the frequency of the individual's gamma brainwaves to align to the frequency of the steady state stimuli and/or the electrical current. As explained in more detail below, the present embodiments may be implemented on an individual while the individual is awake, or while the individual is asleep.

### Sensory Stimulation of the Brain

Neural oscillatory activity in the gamma frequency occurs between 30-100 Hz, and often at or near 40 Hz. Further, gamma activity has an important role in cognition and neurodegenerative diseases like Alzheimer's disease (AD). Inducing gamma activity in the brains of humans with AD, especially at the 40 Hz frequency, may help treat or prevent the onset/progression of AD. Potential mechanisms include direct or indirect effects on amyloid beta and/or tau proteins, as well as effects on microglia cells.

When an individual is exposed to a visual stimulus, such as a flash of light, for example, the brain's response to that stimulus creates an electrical signal known as a visually evoked potential (VEP). These VEPs can be detected using Electroencephalography (EEG). If a visual stimulus is presented at regular intervals at a given frequency, the VEPs will be detected in the EEG at that same frequency.

For example, exposing an individual to a 10 Hz strobe light will result in a VEP repeating 10 times per second. These are known as steady-state visually evoked potentials, or SSVEPs. SSVEPs are robust EEG signals that can often be detected reliably even when the overall EEG data quality is noisy or interrupted by artifacts. SSVEP signal strength is also modulated by the individuals' attention to, and concentration on, the visual stimulus.

Similarly, auditory stimuli can be presented in a repeating fashion to induce an auditory steady-state response (ASSR). In addition, somatosensory stimuli can be presented to induce steady-state somatosensory evoked potentials (SSSEPs).

Presentation of steady-state stimuli in the gamma frequency at one or more of these sensory modalities (i.e., visual, auditory, somatosensory) can induce gamma frequency oscillatory activity in the brain. However, the degree to which gamma activity is induced by these stimuli is modulated by a variety of factors. Such factors include, but are not limited to, the intensity of the stimuli, the attention paid to the stimuli by the individual, and the mental state of the individual subject to the stimuli. In order to enhance the effect of gamma frequency entrainment using steady-state stimuli at one or more modalities, the present embodiments provide a system and method whereby individuals are provided with real-time feedback on their brain's oscillatory activity as measured by EEG.

With one or more embodiments of the present disclosure, an individual is presented with steady-state stimuli at one or more modalities while EEG activity is collected from one or more sensors. The individual may be presented with the sensory stimuli using one or more output devices (e.g., one or more display outputs, speakers, tactile devices, etc.). The individual is then presented with real-time feedback on the strength, or power, of the induced gamma frequency, as measured by EEG. This feedback could be delivered through a video game interface, for example, in which an avatar's behavior is dependent on the power of the induced gamma frequency. The feedback could take any other form in which the individual understands that their oscillatory brain activity at the given frequency is modulating the feedback received. Examples include a simple bar graph of frequency power, or sounds that are influenced by the EEG activity. Such feedback could also be delivered, and may be most effective, in an immersive environment, such as through a virtual reality (VR) experience or an augmented reality (AR) interface.

The individual would then use this feedback to increase gamma activity in their brain, for example by better attending to the presented steady state stimuli. Through a continuous feedback loop, the individual can use this method to promote gamma activity in the brain to a greater extent than what is typically induced by steady state stimuli alone. This will demonstrably improve the therapeutic impact of gamma frequency entrainment for Alzheimer's individuals, and may lead to more persistent effects of treatment. It may also prove beneficial in the prevention of Alzheimer's in at-risk populations.

### Electrical Stimulation of the Brain

In addition to, or in lieu of, the “indirect” sensory stimulation of the individual's, embodiments of the present disclosure are also configured to “directly” stimulate the individual's brain using an electrical current. Techniques for providing such “direct” stimulation include, but are not limited to, a trans-cranial random noise stimulation (tRNS) technique and a transcranial alternating current stimulation (tACS) technique. In both techniques, a low-voltage electrical current is applied directly to the individual's scalp.

With tACS in particular, an oscillating current is applied at a selected frequency to interact with the cortical oscillations of the brain. To accomplish such electrical stimulation, electrodes are typically placed directly on the individual's scalp over a selected area of the brain. An electrical current is then applied in a selected frequency range to entrain neural oscillations, thereby augmenting them.

Similar to the embodiments that utilize sensory stimulation, the individual is presented with real-time feedback on the strength, or power, of the induced gamma frequency, as measured by EEG through, for example, a video game interface and used to control the behavior of an avatar (e.g., based on the power of the induced gamma frequency), or as described above, in any form in which the individual understands that their oscillatory brain activity at the given frequency is modulating the feedback received. As above, the feedback associated with the electrical current stimulation could also be delivered, and may be most effective, in an immersive environment, such as through a virtual reality (VR) experience or an augmented reality (AR) interface.

According to these embodiments, the individual uses this feedback in a continuous feedback loop to increase gamma activity in their brain. More particularly, the individual can use this embodiment to promote the gamma activity in the brain, thereby improving the therapeutic impact of gamma frequency entrainment in individuals afflicted with a disorder such as Alzheimer's. Moreover, such techniques can help make the effects of treatment more persistent.

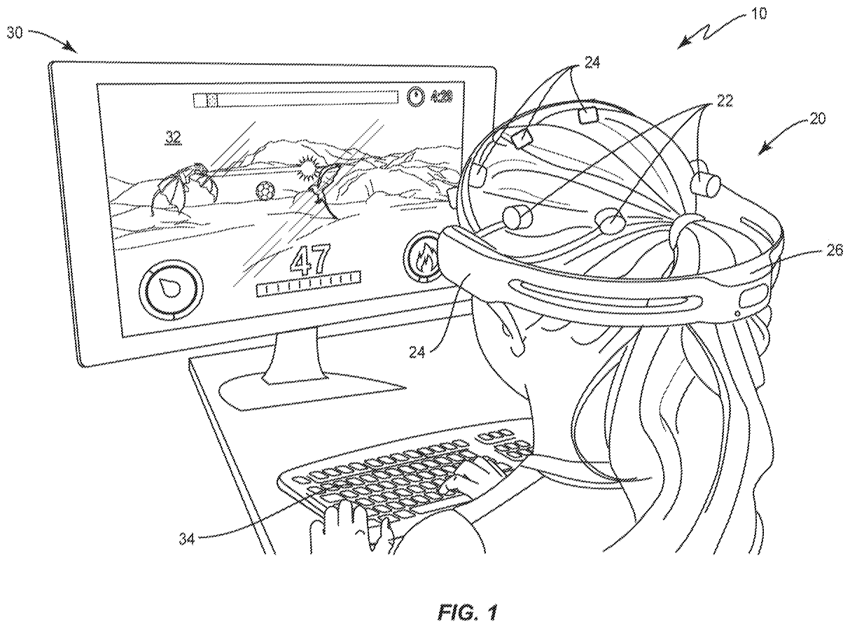

Turning now to the drawings, FIG. 1 illustrates components of a neuro-feedback system 10 configured according to one embodiment of the present disclosure. As seen in FIG. 1, system 10 comprises an EEG device 20 and a computing device 30.

The EEG device 20 is generally configured to measure an individual's brain activity and comprises one or more electrical sensors 22, one or more electrodes 24, and a wireless communications interface 26. In this embodiment, the EEG device 20 is a headset that is worn by the individual. However, those of ordinary skill in the art should readily appreciate that this is for illustrative purposes only, and that the present disclosure is not so limited. In other embodiments, for example, the EEG device 20 may be a helmet, a cap, or headband. In one embodiment, seen in more detail later, EEG device 20 comprises a pair of glasses particularly configured to measure the individual's brain activity while the individual is sleeping. Regardless of its particular structure, however, the EEG device 20 comprises a plurality of embedded or removable electrical sensors 22 configured to measure the individual's brain activity. The sensors 22 and/or the electrodes 24 may use an electrolyte gel or paste to conduct electrical signals from the individual's skin (e.g., the individual's scalp) to the sensor, and from the electrodes to the individual's scalp. Alternatively, sensors 22 and/or electrodes 24 may be a type of dry sensor configured to directly contact the individual's skin.

In operation, the EEG device 20 obtains electrical potential measurements representing the individual's brain activity from the sensors 22, and sends signals representing those measurements in digitized batches to a control application executing on the computing device 30. The electrodes 24 receives a low-voltage electrical current at a selected frequency or frequency range, and applies that electrical current to the individual's scalp. The EEG device 20 also measures the electrical impedance of the sensors 22 as a measure of contact quality, and in some embodiments, is configured to filter out interference from the signals generated by the sensors 22 before those signals are communicated to the computing device 30. Such interference includes, but is not limited to, AC interference generated by nearby power lines and electronic equipment, for example. The control application executing on the computing device 30 continuously processes these received signals, and based on that processing, generates a score value. This score value can then be used by the control application as input into the video game being played by the individual, or into some other computer interface.

Computing device 30 comprises a display device 32 and a user input device, such as a keyboard 34. As stated above, a control application executing on the computing device 30 configures the computing device 30 to receive the signals from sensors 22 and to utilize the incoming signals to enhance the effect of gamma frequency entrainment. Particularly, the control application executing on computing device 30 presents the individual with steady-state stimuli in the gamma frequency at one or more modalities. The control application then receives the resultant EEG signals measured by sensors 22, and controls a video game application, for example, executing on computing device 20 according to the signals received from sensors 22. The control application also provides real-time feedback to the individual indicating the strength or power of the induced frequency as measured by sensors 22. The real-time feedback could take any form needed or desired, including visual, auditory, or both, as long as the feedback enables the individual to understand that their oscillatory brain activity at the given frequency modulates the real-time feedback they receive.

It should be noted that the EEG device 20 and the computing device 30 are seen in the figures and described herein as being separate components. However, the present disclosure is not so limited. In some embodiments, the EEG device 20 and the computing device 30 can comprise a unitary device. By way of example only, a Virtual Reality (VR) head mounted device (HMD) configured to be worn by an individual can comprise both the EEG device 20 and the computing device 30 components. In these embodiments, the VR HMD could be configured to monitor and record the individual's EEG activity using sensors 22, and/or apply a low-voltage electrical current using electrodes 24, generate the feedback information based on the signals representing the EEG activity, and display the feedback information to the user. Additionally, the VR HMD could also comprise a microphone to accept voice input from the individual and/or communicatively connect to one or more other input devices (e.g., joysticks, force balls/tracking balls, controller wands, data gloves, trackpads, on-device control buttons, motion trackers, bodysuits, treadmills, motion platforms, etc.) to accept user input. Moreover, a VR HMD configured according to embodiments of the present disclosure would include one or more output devices configured to output the steady-state stimuli (e.g., one or more display outputs, speakers, tactile devices, etc.) and/or one or more electrodes configured to deliver electrical stimulation to the individual using the tRNS or tACS approaches previously described.

In other embodiments of the present disclosure, the EEG device 20 is included in an Augmented Reality (AR) HMD or a Mixed Reality (MR) HMD. As in the VR HMD embodiment, the AR HMDs and the MR HMDs would be configured to monitor and record the individual's EEG activity, apply a low-voltage current directly to the individual's scalp, generate the feedback information based on the monitored EEG activity, and display the feedback information to the individual, as well as to accept user input and deliver output to the individual, as previously described.

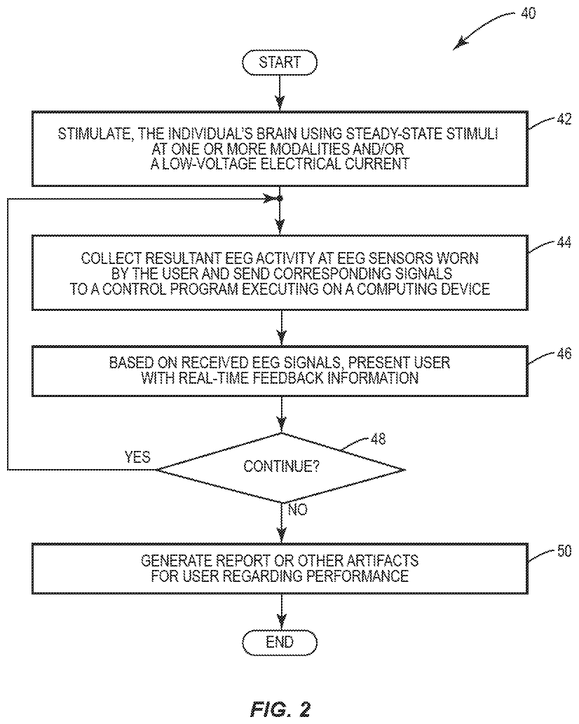

FIG. 2 is a flow diagram illustrating a method 40 for enhancing the effect of gamma frequency entrainment using “direct” stimulation (e.g., electrical current) and/or “indirect” stimulation (e.g., steady-state stimuli) according to one embodiment of the present disclosure. As seen in FIG. 2, method 40 begins by stimulating the individual's brain using the steady-state stimuli at one or more modalities and/or an electrical current to induce gamma activity in the individual's brain (box 42). Many kinds of sensory stimuli can be presented to an individual to induce such gamma activity in the individual. By way of example, the stimuli may be visual stimuli that changes at a predetermined frequency. Such visual stimuli induce an SSVEP and can include, but are not limited to:
a flickering shape or pattern that oscillates between on and off, or between two or more colors such as white and black;
an image that appears before the individual and then disappears, repeatedly;
a point or dot that flashes on/off or between two or more colors;
a series of such points or dots; and
a pattern having colors that invert.

In some embodiments, the SSVEP-inducing visual stimuli could be presented using LED lights, or as images on a screen (e.g., display 32), or through any other method of visual presentation. In other embodiments, however, the SSVEP-inducing visual stimuli could be presented to the individual in a variety of ways via an Augmented Reality (AR) or VR system. For example, the visual stimuli may be presented to the individual:
as elements of a visual field;
as overlaid digital objects;
as elements of a graphical user interface (GUI);
as visual manipulations of real-world elements;
as visual stimuli on the device creating the AR/VR experience (e.g., a head mounted display (HMD) device); or
via other means of visual presentation.

Those of ordinary skill in the art should understand that the present disclosure is not limited solely to the presentation of visual stimuli, and that other embodiments of the present disclosure are configured to present the individual with other types of stimuli. In one embodiment, for example, the individual is presented with auditory stimuli in a repeating fashion to induce an auditory steady-state response (ASSR). ASSRs can be induced in various ways, including, but not limited to, the rendering of repetitive tones or beats through speakers at an intended frequency. In another embodiment, the individual is presented with somatosensory stimuli, such as a surface that vibrates at an intended frequency, to induce steady-state somatosensory evoked potentials (SSSEPs).

In addition, as stated above, some embodiments of the present disclosure are also configured to induce or amplify gamma frequency activity in the individual's brain using a neuromodulatory stimulation technique, such as the transcranial random noise stimulation (tRNS), or the transcranial alternating current stimulation (tACS). With tACS, as previously stated, it is possible to directly modulate an ongoing rhythmic brain activity by applying oscillatory currents on the human scalp.

Additionally, in some embodiments, the present disclosure utilizes a combination of two or more different stimuli types, such as visual and auditory stimuli, or visual, auditory, and somatosensory stimuli, as well as using tRNS or tACS. Thus, the present disclosure is configured to induce and/or amplify gamma frequency oscillatory activity in the individual's brain using any of a variety of direct and/or indirect techniques to enhance and propagate that effect through a feedback loop with the individual.

Regardless of the particular type of direct and/or indirect stimulation that is presented to the individual, however, the stimulation is presented in the gamma frequency at one or more sensory modalities, or through tACS or tRNS, to induce gamma frequency oscillatory activity in the individual's brain. The sensors 22 associated with EEG device 20 detect the EEG activity caused by the stimulation, and send corresponding signals to the control program executing on computing device 30 (box 44). Signals may be sent to computing device 30 using any of a variety of known communication protocols, including but not limited to, BLUETOOTH via the short-range communications interface 26 on EEG device 20.

Responsive to receiving the signals from the sensors 22, the control application executing on computing device 30 presents the individual with real-time feedback information regarding the strength, or power, of the induced gamma frequency activity as measured by EEG device 20 (box 46). Particularly, the control application is configured to compute a value indicating the measured strength or power of the induced gamma frequency activity based on the received EEG signals. The control application then provides real-time feedback information to the individual based, at least in part, on that computed value.

In one embodiment, the control application provides the feedback information via a video game interface. For example, in one embodiment, the speed of a car in the video game is controlled depending on the power of the produced frequency. The higher the power, as indicated by the computed value, the faster the car is controlled to drive. In another embodiment, the height of a flying object in the video game is controlled relative to the computed value. The higher the computed value (i.e., the higher the power), the higher the object is controlled to fly. As the computed power value decreases, which indicates a decrease in gamma frequency activity in the individual's brain, the slower the car becomes or the lower the object flies.

In addition to feedback provided in a video game, the present disclosure can also provide the real-time feedback to the individual in other ways. For example, in one embodiment, the control application is configured to generate and display a simple bar graph of frequency power. With this graph, the bars in the graph could change (e.g., in size or color) relative to the computed value, which changes in response to the increase and decrease of the gamma frequency activity in the individual's brain. Another embodiment of the present disclosure provides the real-time feedback information as one or more audible sounds that are influenced by the EEG activity. According to another embodiment of the present disclosure, the feedback information is utilized to alter or modify one or more parameters associated with the electrical current being applied to the individual via electrodes 24. Further, as previously described, real-time feedback information could be effectively provided to the individual currently in an immersive environment, such as through a VR experience or an AR interface. Those of ordinary skill in the art will appreciate that the real-time feedback of the present disclosure can take any form needed or desired as long as the individual understands that the induced oscillatory activity in their brain at the given frequency controls the real-time feedback they receive.

The method continues so long as the individual is presented with the stimulation (box 48). However, when the method is complete, the control application is configured to generate and output a report, for example, indicating the individual's performance (box 50).

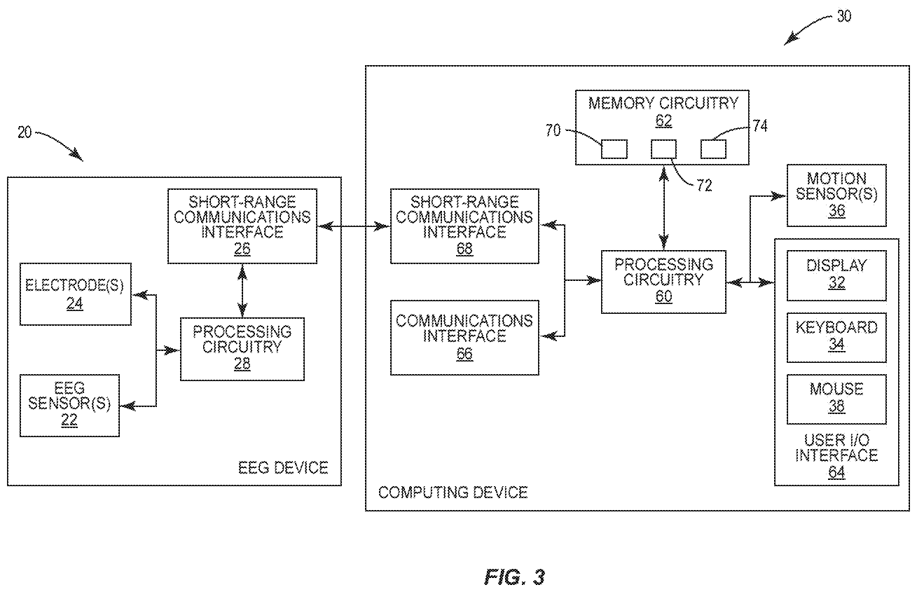

FIG. 3 is a block diagram illustrating some exemplary functional components of an EEG device 20 and a computing device 30. As previously described, EEG device 20 and computing device 30 may be separate components, or they may be combined into a unitary device such as in a VR headset.

As seen in FIG. 3, the EEG device 20 comprises the EEG sensors 22, the electrodes 24, and the short-range communications interface 26, as previously described. Additionally, however, the EEG device 20 may also comprise a processing circuitry 28.

The processing circuitry 28 may comprise, for example, one or more microprocessors, hardware circuits, firmware, or a combination thereof. In the exemplary embodiments disclosed herein, processing circuitry 28 is configured to receive signals from sensors 22 and control the short-range communications interface 26 to transmit those signals to computing device 30 using BLUETOOTH, for example, as previously described. Additionally, the processing circuitry 28 may be configured to filter these signals to remove unwanted interference, as previously described. Further, processing circuitry 28 is configured to cause electrodes 24 to apply a specified low-voltage electrical current to the individual's scalp. In some embodiments, the processing circuitry 28 may incorporate a memory circuit, or have access to a memory circuit, that stores instructions and data to control the functioning of processing circuitry 28.

The computing device 30 comprises a processing circuitry 60, a memory circuitry 62, a user input/output (I/O) interface 64, a communications interface 66, and a short-range communications interface 68. The processing circuitry 60 may be implemented by one or more microprocessors, hardware, firmware, software, or a combination thereof, and generally controls the operation and functions of computing device 30 according to appropriate standards. Such operations and functions include, but are not limited to, executing an application program with which an individual may interact, such as a video game.

Additionally, the processing circuitry 60 may be configured to implement logic and instructions of a control application 70 stored in memory circuitry 62 to perform the functionality of the embodiments of the present disclosure, as previously described. Such functions include, but are not limited to, the receipt of information from the sensors 22 via the short-range communications interface 68, and the filtering of this information, if necessary, to remove unwanted interference, and the delivery of low-voltage electrical currents to the individual's scalp via electrodes 24. In some embodiments, the EEG device 20 analyzes that information to compute the power values, and to input those values into the control application executing on computing device 30 to provide real-time feedback in a loop.

Memory circuitry 62 may comprise any non-transitory, solid state memory or computer readable storage media known in the art. Suitable examples of such media include, but are not limited to, ROM, DRAM, Flash, or a device capable of reading computer-readable storage media, such as optical or magnetic media. The memory circuitry 62 stores programs and instructions, such as the control application 70, that controls the processing circuitry 60 as stated above. In addition, the memory circuitry 62 also stores the application program 72 (e.g., the video game) with which the individual interacts, and in some embodiments, one or more individual profiles 74 comprising information associated with the individual. As previously described, the control application 70 and the application program 72 may be implemented as separate application programs that communicate with each other, or as a single application program.

As seen in FIG. 3, the memory circuitry 62 and the processing circuitry 60 are shown as separate devices that communicate via a bus. However, those of ordinary skill in the art should appreciate that the present disclosure is not limited to this architecture. In other embodiments, the processing circuitry 60 may incorporate the memory circuitry 62, and thus, the two may form a single circuit. Similarly, the present disclosure does not require application program 72 and/or the one or more individual profiles 74 to be stored locally on memory circuitry 62. Rather, in other embodiments, some or all of the application program 72 and the one or more individual profiles 74 are stored on a computer server in the Cloud and streamed to the individual's device.

The User I/O interface 64, as previously stated, comprises components with which the individual can interact with and control the operation of the computing device 30. As seen in FIG. 3, such components include display device 32, keyboard 34, and mouse 38. The communications interface 66 comprises a transceiver or other communications interface that facilitates communications with one or more remote devices over a communications network, such as the Internet and/or a wireless communications network. Those of ordinary skill in the art will appreciate that the communications interface 66 may be configured to communicate with such remote devices using any protocol known in the art.

The short-range communications interface 68 comprises a transceiver configured to transmit data to, and receive data from, the short-range transceiver 26 of EEG device 20. Thus, the short-range communications interface 68 may comprise a BLUETOOTH transceiver that communicates information with the EEG device 20 using the well-known BLUETOOTH protocol. However, other protocols that are known in the art are also suitable for use by the short-range communications interface 68.

In one embodiment, the short-range communications interface 68 comprises a Universal Serial Bus (USB) interface. In these embodiments, the EEG device 20 could comprise a USB device that the individual connects directly to a USB port on the computing device via a USB cable. Thus, the EEG device 20 and the computing device 30 are capable of communicating with each other via both wireless and non-wireless communications means.

The previous embodiments describe the system and method of the present disclosure as being implemented on individuals that are awake. However, those of ordinary skill in the art should readily appreciate that the present embodiments are not so limited. In other embodiments, the present disclosure is implemented to enhance the effect of gamma frequency entrainment while the individuals are asleep.

In more detail, embodiments of the present disclosure enhance the effect of gamma frequency entrainment using one or both of steady-state stimuli at one or more modalities by exposing individuals to steady-state multi-modal stimuli (visual, auditory, and/or somatosensory) and electrical stimulation (tRNS, tACS), at the gamma frequency for an extended period of time while the individuals are asleep. At the same time, the present embodiments collect EEG activity from one or more sensors attached to the individual. The EEG activity measures the strength of the induced gamma activity and/or the propagation of gamma activity in different brain regions while the individual is sleeping. This real-time EEG feedback information on the individual's brain oscillatory activity is then processed and used to drive and optimize the sensory stimulation to maximize gamma entrainment. In particular, embodiments of the present disclosure are configured to automatically adjust one or more parameters associated with the stimuli and/or the electrical current being provided to the sleeping individual based on the feedback. For example, embodiments of the present disclosure may automatically adjust parameters that define the intensity and/or wavelength of light of a visual stimulus, the volume and tone of an auditory stimulus, the strength of vibration in a somatosensory stimulus, and the strength/frequency/protocol/location of tACS stimulation.

In some embodiments, the EEG feedback information is also used to determine the sleep-stage of the individual. Thus, in these embodiments, the EEG device 20 is also configured to measure the individual's other brain waves (i.e., alpha waves, beta waves, delta waves, and theta waves) in addition to the gamma waves, so that the information representing those other signals can be included in the EEG feedback information. The EEG feedback information can then be used as input into a control program that controls the manner in which the stimuli is provided to the individual during sleep. For example, the control program may, in one embodiment, control lights to flash on and off at a specified frequency, control the volume and/or tone of auditory stimuli, control a tactile generator to increase and/or decrease the strength of vibration in a somatosensory stimulus, and/or control one or more of the strength/frequency/protocol/location of tACS stimulation.

Additionally, according to one embodiment, the parameters are automatically adjusted based on the stage of sleep of the individual and the risk of waking. For example, if it is determined that the individual is susceptible to being awakened based on that individual's sleep EEG patterns (i.e., the information representing one or more of the monitored alpha waves, beta waves, delta waves, gamma waves, and theta waves), the stimulation can be terminated or adjusted appropriately to ensure sleep isn't interrupted. Similarly, if it is determined that the individual is in a stage of sleep that's well-suited for gamma entrainment, the stimulation can be adjusted to optimize gamma entrainment. In this manner, the present embodiments use EEG feedback to both optimize the strength of the gamma entrainment, and to automatically adjust one or more parameters associated with the stimuli (and/or the tACS simulation) to adjust the gamma stimulation according to the sleep patterns of individuals while at the same time, mitigating the risk of waking the individual. That is, system 10 is configured according to the present embodiments to provide the most effective stimulation possible to an individual while not waking or interrupting the healthy sleep patterns of the individual.

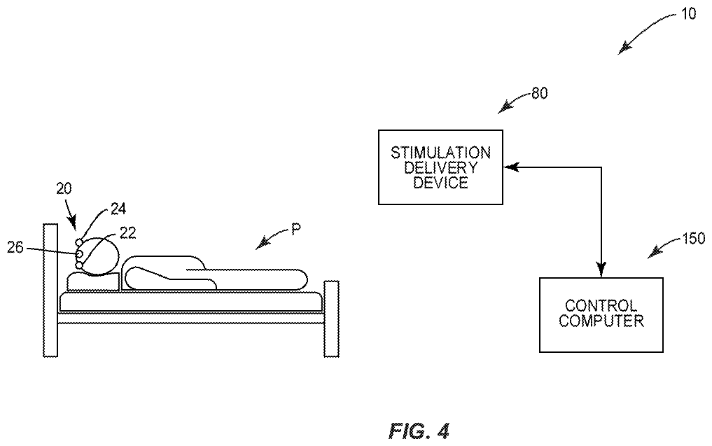

FIG. 4 illustrates components of system 10 configured to provide an individual P with the steady-state stimuli at one or more modalities while the individual P is asleep according to one embodiment of the present disclosure. In this embodiment, system 10 comprises a stimulation delivery device (SDD) 80 for providing one or more of SSVEP, ASSR, SSSEP inducing stimuli, and electrical stimulation (e.g., tRNS, tACS) to a sleeping individual P, an EEG device 20 for measuring the brain activity occurring in various regions of the sleeping individual's brain in response to the stimuli, and a control computer 150 for generating EEG feedback information from the EEG measurement data, and for controlling how and/or when SDD 80 provides the stimuli and/or electrical current based on the generated EEG feedback information.

The SDD 80 is configured to deliver one or more of a visual, auditory, and/or somatosensory stimuli to an individual P who is asleep to induce SSVEPs and/or ASSRs and/or SSSEPs, respectively, and to deliver the electrical current to electrodes 24. For example, in some embodiments, SDD 80 comprises a display device (e.g., a television or computer monitor) that utilizes Light Emitting Diodes (LEDs) to output the visual stimuli to the individual P, and/or a set of loudspeakers, headphones, or ear buds configured to output the auditory stimuli to the individual P, and/or a tactile generating device configured to output somatosensory stimuli to the individual P. In other embodiments, SDD 80 comprises a “smart sleep mask” designed to be worn by the individual P during sleep. As described later in more detail, the smart sleep mask comprises a visual stimulus output component (e.g., one or more LEDs) configured to deliver an SSVEP-inducing visual stimuli to the sleeping individual P, and/or auditory stimulus output components (e.g., headphones or ear buds) configured to deliver an ASSR-inducing auditory stimuli to the sleeping individual P, and/or one or more tactile components configured to deliver a SSSEP-inducing somatosensory stimuli to the sleeping individual P. In addition, or in lieu of such stimuli, SDD 80 can also be configured with one or more electrodes 24 to generate and deliver the electrical stimulation to the individual P, such as used with TENS or tACS. In these embodiments, SDD 80 can generate the electrical stimulation that is delivered to the individual P's scalp via electrodes 24 that are mounted, for example, on EEG device 20.

Regardless of the particular type of SSD 80, however, SSD 80 communicates with control computer 150 via any communications technology known in the art (e.g., BLUETOOTH, wireless, Ethernet, etc.) such that its operation is controlled to provide the visual and/or auditory and/or somatosensory stimuli, and/or electrical stimulation, to a sleeping individual P.

SSD 80 can also provide various different types of SSVEP-inducing and/or ASSR-inducing and/or SSSEP-inducing stimuli to an individual P according to the present disclosure. By way of example only, visual stimuli that induces SSVEP can include, but is not limited to, a flickering shape or pattern that oscillates between on and off, or that changes between two or more colors (e.g., black and white), an image that appears and disappears repeatedly at a predetermined frequency or frequencies, a point or dot that flashes on and off or between two or more colors, a series of such points or dots, a pattern whose colors invert, or any other visual stimulus that changes at a predetermined frequency. For ASSR-inducing embodiments, auditory stimuli can include sounds such as tones, beats, and chirps, and may be modulated at any desired rate and rendered at any desired frequency. SSSEP-inducing somatosensory stimuli can include tactile signals causing the individual to perceive a pattern of touch.

The EEG device 20, as described above, is a headset worn by individual P and is generally configured to measure the individual's P brain activity while he/she is sleeping. As in the previous embodiments, the EEG device 20 obtains electrical potential measurements representing the individual's P brain activity from the sensors 22, and sends signals representing those measurements in digitized batches to a control application executing on control computer 150 using communications interface 26. Additionally, EEG device 20 measures the electrical impedance of the sensors 22 as a measure of contact quality, and filters out interference from the signals generated by the sensors 22 before communicating those signals to the control computer 150. The control application executing on the control computer 150 continuously processes these received signals, and based on that processing, generates EEG feedback information. This EEG feedback information can then be used by the control application to control the amount and type of stimuli provided to the individual P by SSD 80.

Control computer 150, as stated above, executes a control application that configures the control computer 150 to receive signals from EEG device 20, and to utilize those incoming signals to enhance the effect of gamma frequency entrainment. Particularly, the control application executing on control computer 150 provides SDD 80 with SSVEP and/or ASSR and/or SSSEP-inducing stimuli, and controls SDD 80 to output that steady-state stimuli in the gamma frequency at one or more modalities (and/or an electrical current) to individual P while he/she is asleep. The control application then receives the resultant EEG signals measured by sensors 22, and uses those signals to generate “real-time” EEG feedback information. This “real-time” feedback information indicates the brain's oscillatory activity, and is used to drive and optimize the visual and/or auditory and/or somatosensory stimuli and/or the low-voltage electrical current output by SSD 80 to maximize gamma entrainment.

Additionally, as stated above, in some embodiments, the generated EEG feedback information is also used to monitor the sleep-stage of individual P. Monitoring the sleep-stage of the individual P allows the control computer 150 to control SDD 80 to carefully stimulate the individual's brain (i.e., using the visual and/or auditory and/or somatosensory stimuli and/or electrical current) during the optimum stages of sleep, while simultaneously decreasing the risk that the stimulation will wake the individual P prematurely. For example, if based on their sleep EEG patterns, a given individual P appears to be waking, or is susceptible to waking, control computer 150 can terminate or otherwise adjust one or more of the visual, auditory, and somatosensory stimuli, as well as the electrical current, to ensure that the individual's P sleep is not interrupted. As the generated EEG feedback information indicates that individual P is returning to, or entering, a more stable or deeper sleep-stage, control computer 150 can begin to adjust or return the visual, auditory, and/or somatosensory stimuli SDD 80 to previous levels. Similarly, if the monitored sleep-stage indicates that the given individual P is in a stage of sleep that's well-suited for gamma entrainment, control computer 80 can adjust the one or more of the visual, auditory, and somatosensory stimuli, as well as the electrical current, accordingly to optimize the strength of the gamma entrainment.

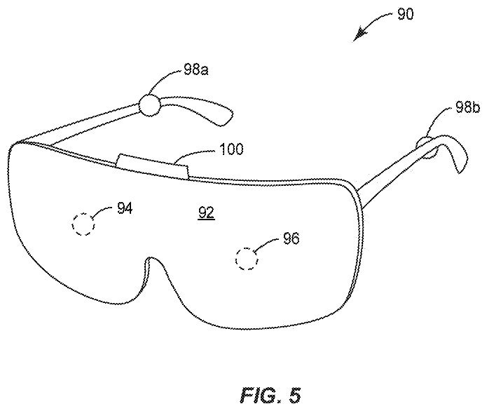

FIG. 5 is a perspective view of a type of SSD 80 suitable for use with embodiments of the present disclosure. In this embodiment, SSD 80 comprises a “smart sleep mask” 90 that is worn by the individual P while sleeping. However, this is for illustrative purposes only. Although the SSD 80 of FIG. 5 is shown as a mask 90, other embodiments of the present disclosure provide the SSD 80 in the form of a headset used for VR/AR/MR.

As seen in FIG. 5, mask 90 comprises a lightweight shade panel 92, one or more LEDs 94, 96 disposed in or on the shade panel 92, a pair of speakers 98a, 98b (collectively, speakers 98) disposed on a frame of the mask 90, and a communications interface circuitry 100. Shade panel 92 is designed to completely cover the eyes of the individual P when he/she is wearing mask 90. In use, shade panel 92 blocks unwanted ambient light, but is controlled to provide its own light using the one or more LEDs 94, 96. In particular, LEDs 94, 96 are controlled by control computer 150 to deliver SSVEP-inducing visual stimuli to individual P during sleep. For example, according to the present disclosure, LEDs 94, 96 can be controlled by control computer 150 to show one or more of the following SSVEP-inducing stimuli:
a flickering shape or pattern that oscillates between on and off;
a flickering shape or pattern that changes between two or more colors (e.g., black and white);
an image that repeatedly appears and disappears at a predetermined frequency or at varying frequencies;
a point or dot, or a series of points or dots, that flashes between two or more colors;
a pattern of light(s) in which the colors of the lights invert at a predetermined frequency or frequencies; or
any other visual stimulus that changes at a predetermined frequency.

In embodiments designed to induce ASSR, speakers 98 deliver the auditory stimuli to the individual's P ears. Such auditory stimuli include sounds such as tones, beats, and chirps, and may be modulated at any desired rate and rendered at any desired frequency. In one embodiment, speakers 98 comprise a set of ear buds that insert into individual's P ears, while in other embodiments, speakers 98 comprise a set of loudspeakers that sit over the ears of individual P.

Regardless, the operation of the LEDs 94, 96 and the speakers 98 are controlled by the control computer 150. In particular, mask 90 is communicatively connected to control computer 150 via a communications link. In one embodiment, the communication link comprises a wireless communication link, such as a BLUETOOTH link or WiFi link. In these embodiments, communications interface circuitry 100 comprises a suitable wireless communications circuit, as is described in more detail later. In other embodiments, however, the communication link comprises a hardwire link. Such hardwire links may comprise an Ethernet cable, for example, or any other physical wires and/or cables connecting mask 90 to control computer 150. In these latter embodiments, communications interface circuitry 100 comprises a port or jack configured to physically releasably receive an Ethernet cable or similar cable. In still other embodiments, the mask 90 is also configured to implement the functions of the control computer 150. Therefore, according to the present disclosure, mask 90 and control computer 150 can comprise a unitary device.

In operation, communications interface circuitry 100 receives control signals from control computer 150. Upon receipt, the control signals are processed by processing circuitry in the communications interface circuitry 100 and are used to control the LEDs 94, 96 and/or speakers 98 to provide the SSVEP and/or ASSR inducing stimuli previously described. In some embodiments, although not explicitly seen in FIG. 5, mask 90 may also include electrodes 24. In these cases, the smart sleep mask 90 may be configured to deliver a controlled low-voltage electrical current to the individual's scalp as previously described.

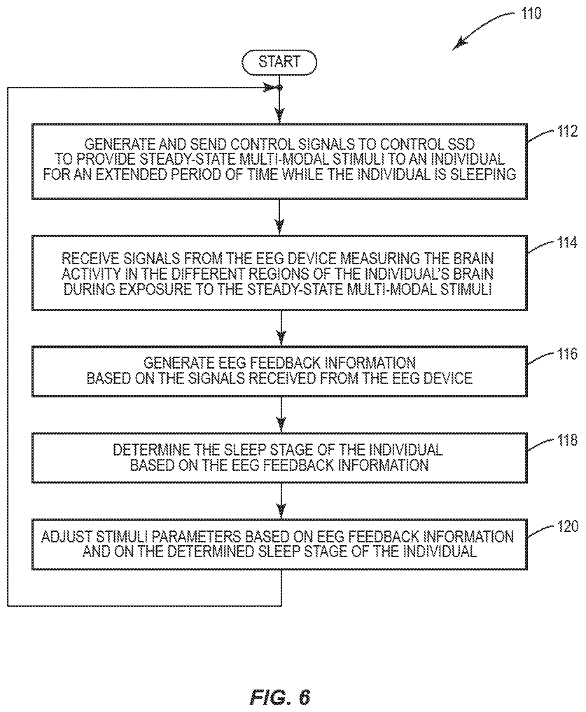

FIG. 6 is a flow diagram illustrating a method 110 for treating and preventing cognitive disorders in an individual through gamma entrainment while the individual P is asleep. Such cognitive disorders may include, but are not limited to, Alzheimer's disease. For ease of discussion, the SDD 80 providing the SSVEP and/or ASSR-inducing stimuli to the individual P in method 110 is the mask 90. However, this is for illustrative purposes only. Those of ordinary skill in the art will readily appreciate that method 110 is equally as suitable for use when SDD 80 comprises a display device, such as a television or computer monitor, and/or a tactile generator. Additionally, according to embodiments of the present disclosure, mask 90 can be configured to include one or more electrodes 24. As stated above, mask 90 can be configured to deliver the electrical stimulation associated with tACS-based approaches directly to the scalp of the individual. Thus, it is understood that method 110 can be used to stimulate the individual's brain using one or both of sensory stimulation (i.e., one or more steady-state stimuli) and an electrical current.

As seen in FIG. 6, method 110 begins with the control computer 150 generating the control signals used to control the visual and/or auditory output components (e.g., LEDs 94, 96 and/or speakers 98) to output the SSVEP and/or ASSR inducing stimuli to the individual P while that individual P is asleep, and sending those control signals to mask 90 (box 112). Mask 90, upon receipt of the control signals, controls LEDs 94, 96 and/or speakers 98 to output the stimuli steady-state stimuli. The EEG device 20, meanwhile, measures the brain activity of the sleeping individual P and sends signals representing that brain activity back to control computer 150. As previously stated, the EEG device 20 measures the brain activity in one or more different regions of the individual's P brain. Control computer 150 receives the signals from the EEG device 20 (box 114) and generates EEG feedback information based on those signals (box 116). Then, based on the generated EEG feedback information and/or on the raw signals received from EEG device 20, control computer 150 determines the sleep-stage of the individual P (box 118), and automatically adjusts the stimuli parameters (box 120). In this embodiment, the stimuli parameters are adjusted based on the EEG feedback information and on the determined sleep-stage of the individual P.

Using method 110, control computer 150 is configured to generate and utilize the EEG feedback information to both optimize a strength of the gamma entrainment, as well as to synchronize the gamma stimulation parameters to align with the sleep patterns of the individual P.

As stated above, the present disclosure provides other methods for inducing gamma and/or amplifying activity in the individual's P brain. Such methods include, but are not limited to, the previously described tRNS and tACS. By way of example, one embodiment of the present disclosure employs a tACS-based approach to electrically stimulate selected areas or regions of the individual's P brain. With this embodiment, as previously stated, one or more tACS parameters (e.g., voltage, frequency, localization, and the like) are adjusted or modified in “real-time” (i.e., responsive to receiving real-time EEG feedback information) to maximize gamma activity in the individual's P brain. According to the present disclosure, these embodiments may be performed regardless of whether the individual is awake or asleep. In one particular embodiment, the tACS stimulation is applied during a training period while the individual P is awake as a complement or alternative to other types of sensory stimulation approaches, such as audio-visual stimulation, with one or more parameters controlling the stimulation being modified in real-time based on the EEG feedback information.

Additionally, some embodiments of the present disclosure utilize machine learning techniques to optimize the parameters on which the stimulation is based to maximize the gamma activity in the individual's brain, and/or maximize an individual's P performance on a regularly-performed cognitive assessment, for example. Machine learning techniques are beneficial because they allow a device implementing the method (e.g., control computer 150) to optimize the stimulation parameters on a per-individual basis.

In more detail, the parameters being changed are dynamic and will often change very quickly. Further, parameters that are optimized for a first individual P₁ may not be optimized for a second individual P₂. This is because different individuals typically respond differently to the stimulation. That is, the first individual P₁ may respond very favorably to stimuli and/or an electrical current optimized according to the first set of optimized parameters while the second individual P₂ responds best to the same stimuli and/or an electrical current driven by a different set of optimized parameters. By using a machine-learning approach, the present embodiments are able to “learn” which parameters and/or stimuli and/or electrical current work best for a particular individual P, and utilize those parameters when stimulating the brain of the particular individual P.

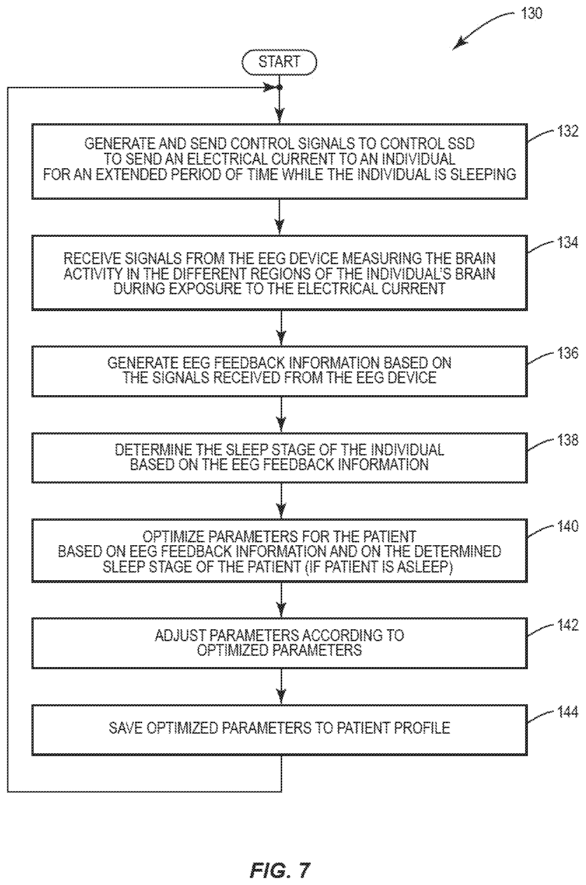

FIG. 7 is a flow diagram illustrating a method 130 for treating cognitive disorders in an individual using tACS to induce and/or amplify the gamma activity in the individual's P brain. In this embodiment, the individual P is fitted with a device, such as EEG device 20 or mask 90, for example, having one or more electrodes 24 that contact the individual's P scalp directly. As is known in the art, the electrodes are placed over an area of interest on the individual's P scalp and are associated with a reference electrode disposed over a neutral location of the individual's P scalp. During treatment, an oscillating sinusoidal current is applied to the individual P via the electrodes 24 at a selected frequency.

Method 130 begins with control computer 150 generating the control signals used to control the SSD 80 to provide the electrical current to the individual P (box 132). In this embodiment, SSD 80 generates and sends an oscillating sinusoidal current at a selected frequency to the electrodes contacting the individual's scalp. In addition, as stated above, mask 90 may also generate the control signals needed to control LEDs 94, 96 and/or speakers 98 to output audio-visual stimuli steady-state stimuli. The EEG device 20, meanwhile, measures the brain activity in one or more different regions of the individual's P brain and sends signals representing that brain activity back to control computer 150, as previously described. Control computer 150 receives the signals from the EEG device 20 (box 134) and generates EEG feedback information based on those signals, as previously described (box 136). Then, in cases where the individual P is asleep, control computer 150 determines the sleep-stage of the individual P based on the generated EEG feedback information and/or on the raw signals received from EEG device 20 (box 138). In cases where the individual is awake, the step of determining the sleep-stage need not be performed.

Method 130 then optimizes the stimulation parameters based on the EEG feedback information, and if the individual is sleeping, on the determined sleep-stage of the individual P (box 140). In particular, method 130 automatically selects the parameters that are to be adjusted, and determines how those parameters are to be adjusted. Method 130 then adjusts the selected parameters accordingly (box 142), and based on the adjusted parameters, generates the control signals needed to continue delivering the electrical current (and/or the steady-state stimuli) to the individual P (box 132). Selecting and adjusting the parameters in this manner affects the electrical current being output to the individual, thereby optimizing the gamma frequency oscillatory activity induced by the electrical current. Additionally, because the parameters are optimized for a given individual, method 130 also calls for saving the optimized parameters to an individual profile (box 144). Saving the optimized parameters on a per-individual basis allows for the easy and quick customization of treatment for the given individual.

There are a variety of machine learning techniques suitable for use in optimizing the parameters associated with the steady-state stimuli and/or the electrical current stimulation according to the embodiments of the present disclosure. In one embodiment, however, control computer 150 employs any of a known variety of optimization algorithms to select parameters associated with the steady-state stimuli, and to adjust those parameters based on the efficacy of the current parameters to produce the desired gamma activity in the individual's brain.

By way of example only, one embodiment of the present disclosure uses a genetic algorithm with a neural network algorithm to select and adjust the parameters. Genetic algorithms are a sub-class of evolutionary algorithms, and are designed to evolve a plurality of candidate solutions towards a selected solution. In the context of the present disclosure, the genetic algorithm receives the feedback information, as well as information regarding the individual's sleep-stage, and produces a result indicating which parameters should be adjusted, as well as how those parameters should be adjusted. For example, in one embodiment, control computer 150 is configured to adjust the frequency of the oscillating current, the voltage of the oscillating current, the location on the individual's P scalp to which the current is applied, and/or the periodicity at which the oscillating current is delivered to the individual's scalp based on a result produced by the genetic algorithm. In another embodiment, the parameters are selected and adjusted based on the results of a comparison of the EEG feedback information for the given individual to previously collected and stored EEG information (e.g., baseline information) for that individual P.

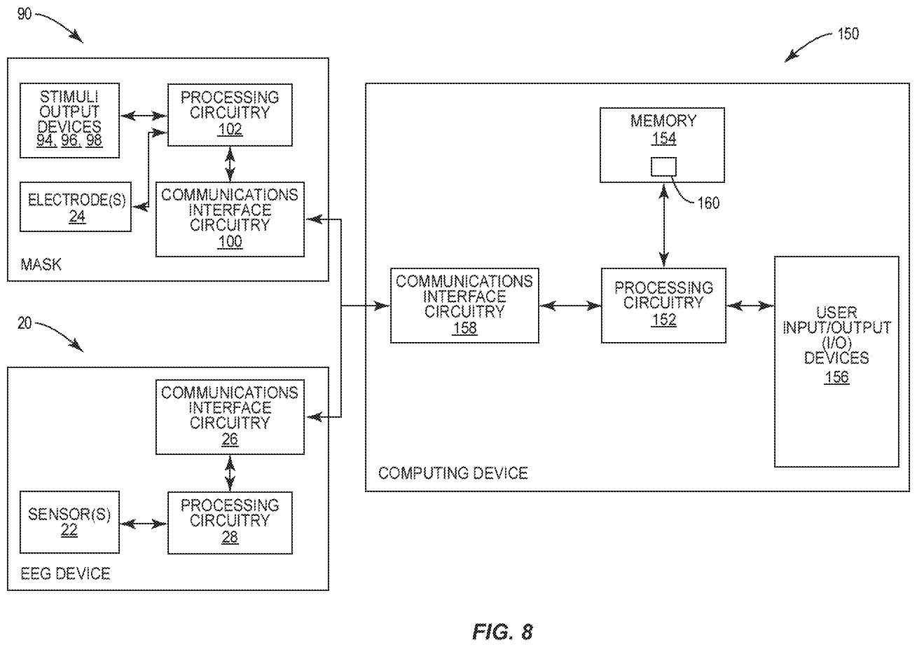

FIG. 8 is a functional block diagram illustrating some exemplary functional components of system 10 including EEG device 20, mask 90, and control computer 150. The EEG device 20 comprises the same or similar components (i.e., EEG sensors 22, communications circuitry 26, and processing circuitry 28) and functions as previously described.

Mask 90 comprises a “smart sleep mask” that is worn by the individual P while asleep, and comprises one or more stimuli output devices (e.g., LEDs 94, 96 and/or speakers 98), electrodes 24, communications interface circuitry 100, and processing circuitry 102. In operation, communications interface circuitry 100, which may comprise a BLUETOOTH communications interface, for example, is configured to receive and process the raw control signals from control computer 150. Processing circuitry 102, which may comprise one or more microprocessors, hardware circuits, firmware, or a combination thereof, then processes those raw control signals. In one embodiment, for example, the raw control signals comprise one or more messages identifying particular visual and/or auditory components (e.g., LEDs 94, 96 and speakers 98), as well as a particular pattern for the stimuli. Processing circuitry 102 then generates one or more signals to control the operation of LEDs 94, 96 and/or speakers 98 such that these components provide corresponding respective SSVEP and ASSR inducing stimuli to the individual P while he/she is asleep.

As stated above, the present embodiments are not limited to the use of a smart sleep mask 90, which in this embodiment, are formed as glasses. Rather, some embodiments utilize a headset associated with a VR/AR/MR environment. Therefore, the components illustrated in FIG. 8 with respect to mask 90 are also the components associated with a VR/AR/MR headset used in at least one embodiment of the present disclosure.

Control computer 150 comprises a processing circuitry 152, a memory circuitry 154, a user input/output (I/O) interface 156, and communications interface circuitry 158. The processing circuitry 152 may be implemented by one or more microprocessors, hardware, firmware, software, or a combination thereof, and generally controls the operation and functions of control computer 150 according to appropriate standards. Such operations and functions include, but are not limited to, executing a control program 160 stored in memory 154 to control mask 90 to provide steady-state SSVER and/or ASSR and/or SSSEP-inducing stimuli at one or more modalities to a sleeping individual P, while simultaneously collecting EEG signals indicating the individual's P brain activity, as previously described. In some embodiments, the logic and instructions comprising control program 160 configured control computer 150 to analyze signals received from the EEG device 20 to determine the sleep-stage of the individual P, and to adjust the parameters of the SSVEP and/or ASSR and/or SSSEP-inducing stimuli, and/or tACS stimulation, accordingly.

Memory 154 may comprise any non-transitory, solid state memory or computer readable storage media known in the art. Suitable examples of such media include, but are not limited to, ROM, DRAM, Flash, or a device capable of reading computer-readable storage media, such as optical or magnetic media. The memory 154 stores programs and instructions, such as the control program 160, that controls the processing circuitry 152 as stated above.

As seen in FIG. 8, the memory 154 and processing circuitry 152 are shown as separate devices that communicate via a bus. However, those of ordinary skill in the art should appreciate that the present disclosure is not limited to this architecture. In other embodiments, the processing circuitry 152 may incorporate the memory 154, and thus, the two may form a single circuit.

The User Input/Output (I/O) devices comprise components with which the individual can interact with and control the operation of control computer 150. While not expressly seen in FIG. 8, such components include, but are not limited to, a display device, a keyboard, and a mouse.

The communications interface circuitry 158, in this embodiment, comprises a transceiver or other communications interface that facilitates communications with the mask 90 and EEG device 20. For example, communications interface circuitry 158 communicates with the communications interface circuits 16, 100 of EEG device 20 and mask 90, respectively, using the well-known BLUETOOTH protocol. In other embodiments, however, communications interface circuits 26, 100, 158, comprise transceivers configured to communicate over much longer distances. In these latter embodiments, communications interface circuits 26, 100, 158 may be configured to communicate with each other over a network such as the Internet and/or a wireless communications network. In other embodiments, communications interface circuitry 158 comprises a Universal Serial Bus (USB) interface. In these embodiments, EEG device 20 and/or mask 90 could comprise USB devices that connect directly to a USB port on the computing device via a USB cable. Thus, one or both of EEG device 20 and mask 90, as well as control computer 150, are capable of communicating with each other via wireless and non-wireless communications means.

The present disclosure may, of course, be carried out in ways other than those specifically set forth herein without departing from essential characteristics of the disclosure. For example, the present embodiments describe the system 10 and its various components and methods as being useful for treating individuals with Alzheimer's disease. However, this is for illustrative purposes only, as the present embodiments are not so limited. In some embodiments, the system of the present disclosure is used to treat individuals having other types of dementia and/or cognitive dysfunctions.

Additionally, the embodiments disclosed herein utilize a non-invasive EEG device 20 to measure the signals associated with brain activity. However, this is for illustrative purposes only. EEG is not the only technique with which the present disclosure is able to detect signals associated with such brain activity. In other embodiments, the present disclosure utilizes invasive techniques to detect such signals. These include, but are not limited to, implants or other devices that directly contact the brain of an individual.

Further, according to the present embodiments, the EEG device 20 is not only configured to detect steady-state evoked potentials, as previously described. Rather, EEG device 20 can also be configured to measure the gamma frequency oscillatory activity induced or amplified in a tACS-based embodiment. Additionally, EEG device 20 can be used to monitor other types of brain waves as well, such as the individual's alpha waves, beta waves, delta waves, and theta waves.

Regardless of whether a particular device is invasive or non-invasive, however, EEG device 20 measures signals to detect the individual's P brain activity regardless of whether the individual P is awake or asleep, as previously described.

Similarly, the present disclosure is not limited to presenting the individual with feedback information regarding the strength, or power, of the induced gamma frequency activity. In some embodiments, for example, the present disclosure provides feedback information regarding the extent of the gamma frequency neuronal stimulation. Therefore, the present embodiments are to be considered in all respects as illustrative and not restrictive, and all changes coming within the meaning and equivalency range of the appended claims are intended to be embraced therein.

What is claimed is:

1. A method for treating an individual with a cognitive disorder, the method comprising:
stimulating an individual's brain using one or both of sensory stimulation and an electrical current; and
while the individual's brain is being stimulated:
receiving signals indicating gamma frequency oscillatory activity in the individual's brain induced by the stimulation;
generating feedback information based on the signals indicating the gamma frequency oscillatory activity in the individual's brain;
outputting the feedback information in a feedback loop; and
enhancing entrainment of gamma frequency induced by the stimulation in the individual's brain responsive to the feedback information in the feedback loop.

2. The method of claim 1 wherein stimulating an individual's brain using one or both of sensory stimulation and an electrical current comprises outputting one or more steady-state stimuli at one or more modalities to the individual.

3. The method of claim 2 wherein outputting one or more steady-state stimuli at one or more modalities to the individual comprises outputting the one or more steady-state stimuli in the gamma frequency via a video game interface.

4. The method of claim 3 wherein enhancing entrainment of gamma frequency induced by the stimulation comprises altering a frequency at which the one or more steady-state sensory stimuli is output to the individual based on the feedback information in the feedback loop.

5. The method of claim 3 wherein the signals indicating the gamma frequency oscillatory activity comprise Electroencephalography (EEG) signals measured by an EEG device attached to the individual, and wherein the feedback information comprises data indicating a strength of the gamma frequency oscillatory activity in the individual's brain as indicated by the EEG signals.

6. The method of claim 5 wherein outputting the feedback information in a feedback loop comprises generating one or more input signals to control a behavior of an avatar displayed on the video game interface based on the strength of the induced gamma frequency activity.

7. The method of claim 1 wherein enhancing entrainment of gamma frequency induced by the stimulation comprises:
generating a score value for a video game being played by the individual based on the feedback information; and
inputting the score value into the video game to control play of the video game.

8. The method of claim 1 wherein the method is performed while the individual is asleep.

9. The method of claim 1 further comprising:
determining a sleep-stage of the individual based on the feedback information;
automatically adjusting one or more parameters associated with the one or both of the sensory stimulation and the electrical current being used to stimulate the individual's brain based on the feedback information and the determined sleep-stage; and
while the individual remains asleep, continuing to stimulate the individual's brain using the one or both of the sensory stimulation and the electrical current according to the adjusted one or more parameters without interrupting the sleep-stage of the individual.

10. The method of claim 9 further comprising terminating the stimulation of the individual's brain responsive to determining that the stimulation is interrupting the sleep-stage of the individual.

11. The method of claim 1 further comprising optimizing the gamma frequency oscillatory activity induced by the one or both of the sensory stimulation and the electrical current, wherein optimizing the gamma frequency oscillatory activity comprises:
selecting a set of one or more parameters for the one or both of the sensory stimulation and the electrical current based on a result of an optimization algorithm;
adjusting the set of one or more parameters based on the result of the optimization algorithm.

12. The method of claim 1 wherein enhancing entrainment of the gamma frequency further comprises applying the electrical current to one or more areas of the individual's brain to induce or amplify the gamma frequency oscillatory activity in the individual's brain.

13. The method of claim 1 wherein the method is performed in at least one of a Virtual Reality (VR) environment, an Augmented Reality (AR) environment, and a Mixed Reality (MR) environment.

14. A computing device for treating an individual with a cognitive disorder, the computing device comprising:
communications interface circuitry configured to with an Electroencephalography (EEG) device attached to an individual being treated for a cognitive disorder; and
processing circuitry operatively coupled to the communications interface circuitry, and configured to:
stimulate an individual's brain using one or both of sensory stimulation and an electrical current; and
while the individual's brain is being stimulated:
receive EEG signals indicating gamma frequency oscillatory activity in the individual's brain induced by the stimulation;
generate feedback information based on the EEG signals indicating the gamma frequency oscillatory activity in the individual's brain;
output the feedback information in a feedback loop; and
enhance entrainment of gamma frequency induced by the stimulation in the individual's brain responsive to the feedback information in the feedback loop.

15. The computing device of claim 14 wherein to stimulate the individual's brain, the processing circuitry is configured to output one or more steady-state stimuli at one or more modalities in the gamma frequency via a video game interface.

16. The computing device of claim 15 wherein the processing circuitry is further configured to modify a frequency at which the one or more steady-state sensory stimuli is output to the individual based on the feedback information in the feedback loop.

17. The computing device of claim 14 wherein the feedback information comprises data indicating a strength of the gamma frequency oscillatory activity in the individual's brain as indicated by the EEG signals, and wherein to output the feedback information in the feedback loop, the processing circuitry is configured to generate one or more input signals based on the strength of the induced gamma frequency activity to control a behavior of an avatar displayed on the video game interface.

18. The computing device of claim 14 wherein the individual is asleep during treatment, and wherein while the individual is asleep, the processing circuitry is further configured to:
determine a sleep-stage of the individual based on the feedback information;
automatically adjust one or more parameters associated with the one or both of the sensory stimulation and the electrical current being used to stimulate the individual's brain based on the feedback information and the determined sleep-stage; and
while the individual remains asleep, continuing to stimulate the individual's brain using the one or both of the sensory stimulation and the electrical current according to the adjusted one or more parameters without interrupting the sleep-stage of the individual.

19. The computing device of claim 18 wherein the processing circuitry is configured to terminate the stimulation of the individual's brain responsive to determining that the stimulation is interrupting the sleep-stage of the individual.

20. The computing device of claim 18 wherein the processing circuitry is further configured to:
select a set of one or more parameters for the one or both of the sensory stimulation and the electrical current based on a result of an optimization algorithm;
adjust the set of one or more parameters based on the result of the optimization algorithm; and
continue stimulating the individual's brain using the one or both of the sensory stimulation and the electrical current according to the adjusted set of one or more parameters to optimize the gamma frequency oscillatory activity induced in the brain of the individual by the steady-state stimulus.

21. The computing device of claim 20 wherein the processing circuitry is further configured to apply the electrical current to one or more areas of the individual's brain to induce or amplify the gamma frequency oscillatory activity in the individual's brain.

22. The computing device of claim 14 wherein the computing device comprises one of a Virtual Reality (VR), Augmented Reality (AR), and Mixed Reality (MR) headset.

23. The computing device of claim 22 wherein the one of the VR, AR, and MR headset further comprises the EEG device.

24. A non-transitory computer-readable medium having a control application program stored thereon that, when executed by processing circuitry of a computing device, causes the computing device to:
stimulate an individual's brain using one or both of sensory stimulation and an electrical current; and
while the individual's brain is being stimulated:
receive EEG signals indicating gamma frequency oscillatory activity in the individual's brain induced by the stimulation;
generate feedback information based on the EEG signals indicating the gamma frequency oscillatory activity in the individual's brain;
output the feedback information in a feedback loop; and
enhance entrainment of gamma frequency induced by the stimulation in the individual's brain responsive to the feedback information in the feedback loop.
